(19) United States
(12) Patent Application Publication
(10) Pub. No.: US 2022/0268737 A1
(43) Pub. Date: Aug. 25, 2022

(54) Method, Equipment and Readable Medium for Evaluating Structural Strength of Fiber and Nanosized Materials Reinforced Concrete

(71) Applicant: Zhengzhou University of Aeronautics Ltd., Zhengzhou (CN)

(72) Inventors: Han Li, Zhengzhou (CN); Ke Shi, Zhengzhou (CN); Qiaoyan Guan, Zhengzhou (CN); Qian Zhu, Zhengzhou (CN); Xiaopeng Xie, Zhengzhou (CN)

(21) Appl. No.: 17/564,808

(22) Filed: Dec. 29, 2021

Related U.S. Application Data
(63) Continuation of application No. PCT/CN2021/079961, filed on Oct. 3, 2021.

(30) Foreign Application Priority Data
Feb. 22, 2021 (CN) 202110199176.4

(51) Int. Cl. G01N 29/06; G10K 11/24; G01N 29/07; G01N 33/38

(52) U.S. Cl. CPC G01N 29/069; G10K 11/24; G01N 29/07; G01N 33/383; G01N 2291/102; G01N 2291/011; G01N 2291/018; G01N 2291/0232; G01N 2291/0289

(57) ABSTRACT

A method, an equipment, and a computer readable medium for evaluating structural strength of fiber and nanosized materials reinforced concrete are provided. The method includes: obtaining a sound velocity of a fiber and nanosized materials reinforced concrete test block; deriving a structural compressive strength of the fiber and nanosized materials reinforced concrete test block according to the sound velocity and a relation between fiber and nanosized materials reinforced concrete structural compressive strength and fiber and nanosized materials reinforced concrete sound velocity; determining strengths of various parts of the fiber and nanosized materials reinforced concrete test block based on measured parameters and recorded design parameters of the test fiber and nanosized materials reinforced concrete, and evaluating deviations of the strengths of the respective parts from the structural compressive strength. The partial design strengths and the deviations from the structural compressive strength may be evaluated and corrective measures may be given.

### TECHNICAL FIELD

The disclosure belongs to the technical field of concrete detection, and in particular to a structural strength evaluation method, an equipment and a computer readable medium of fiber and nanosized materials reinforced concrete.

### BACKGROUND

The compressive strength of fiber and nanosized materials reinforced concrete is obtained in experiments. Cubic specimen with side length of 150 mm is used as the standard size specimen of compressive strength of fiber and nanosized materials reinforced concrete below the latest Chinese standard C60 strength. In accordance with the Standard for Mechanical Properties of Ordinary Concrete GB/T50081-2002, the ultimate compressive strength measured by the standard test method is called the standard cube compressive strength of concrete when the cube with a side length of 150 mm is cured under standard curing (temperature 20±2° C., relative humidity above 95%) conditions for 28 days.

According to different principles, the strength testing of fiber and nanosized materials reinforced concrete structures can be generally classified into destructive testing technology and nondestructive testing technology. Among them, although the test results of destructive testing technology are intuitive and reliable, it will cause local damage to the structure, and the tested structure needs to be repaired accordingly, which is not conducive to the later development and maintenance of fiber and nanosized materials reinforced concrete components. On-the-spot testing of the strength of fiber and nanosized materials reinforced concrete structures generally adopts non-destructive testing technology, which has become one of the assessment and analysis methods of engineering accidents. Therefore, non-destructive testing technology plays an important role in the whole construction, check & acceptance and use process. Non-destructive testing technology of fiber and nanosized materials reinforced concrete structure refers to the testing technology that measures one or some physical quantities directly on the structure or component without destroying the fiber and nanosized materials reinforced concrete structure, and infers the strength and other indexes of fiber and nanosized materials reinforced concrete through the correlation between these physical quantities and strength, including ultrasonic method, rebound method, ultrasonic rebound method impact echo method, radar method, infrared imaging method, etc. Among them, the rebound method is widely used because of its simple construction, easy to carry the instrument, simple testing method, high efficiency, low costs and expense, and the shape and size of the object to be measured are generally not restricted, etc. Because it is especially suitable for random and large number of tests on the strength of structural fiber and nanosized materials reinforced concrete at construction sites, it has been recognized by the international academic community as the basic nondestructive testing of fiber and nanosized materials reinforced concrete and the common method of inspection and acceptance of on-site structural fiber and nanosized materials reinforced concrete. After years of research and the accumulation of a large number of laboratory and field data, the national unified strength test curve has been established, and the Technical Specification for Testing Compressive Strength of Concrete by Rebound Method (JGJ/T23-2011) has also been formed. Most regions have also studied and established regional rebound strength test curves suitable for local testing, which provides a basis for quality testing and evaluation of solid projects. However, in the above technologies, there is a lack of an integrated method of strength detection and evaluation.

### SUMMARY

The technical problem to be solved by the disclosure is to provide a method, an equipment and a readable medium for evaluating structural strength of fiber and nanosized materials reinforced concrete, which can evaluate the design strength of each part and the deviation from the structural tensile strength, and provide corrective measures.

To achieve the above purpose, the disclosure adopts the following technical solution:

Specifically, a structural strength evaluation method of fiber and nanosized materials reinforced concrete, including:

S1: obtaining a sound velocity of a fiber and nanosized materials reinforced concrete test block;

S2: deriving a structural compressive strength of the fiber and nanosized materials reinforced concrete test block according to the sound velocity of a fiber and nanosized materials reinforced concrete test block and a relation between a fiber and nanosized materials reinforced concrete structural compressive strength and a fiber and nanosized materials reinforced concrete sound velocity;

S3: determining strengths of various parts of the fiber and nanosized materials reinforced concrete test block based on measured parameters and recorded design parameters of the fiber and nanosized materials reinforced concrete test block, and evaluating deviations of the strengths of the respective parts from the structural compressive strength.

In an embodiment, the sound velocity of the fiber and nanosized materials reinforced concrete test block is obtained based on an acoustic impedance method, specifically including the following steps:

coupling a sound source with a sound transmission medium under a condition that the sound transmission medium is not in contact with the fiber and nanosized materials reinforced concrete test block, controlling the sound source to transmit a first sound wave signal with predetermined frequency, amplitude and waveform to the sound transmission medium, and receiving a first echo amplitude of the first sound wave signal after being reflected by an interface between the sound transmission medium and the air back to reach the sound source;

coupling the sound source with the sound transmission medium under a condition that the sound transmission medium is in contact with the fiber and nanosized materials reinforced concrete test block, controlling the sound source to transmit a second sound wave signal with the with predetermined frequency, amplitude and waveform to the sound transmission medium, and receiving a second echo amplitude of the second sound wave signal after being reflected by an interface between the sound transmission medium and the fiber and nanosized materials reinforced concrete test block back to reach the sound source;

calculating an acoustic pressure reflection coefficient of the interface between the sound transmission medium and the fiber and nanosized materials reinforced concrete test block according to the first echo amplitude and the second echo amplitude; and

calculating a characteristic impedance of the fiber and nanosized materials reinforced concrete test block according to the acoustic pressure reflection coefficient, and calculating the sound velocity of the fiber and nanosized materials reinforced concrete test block according to the characteristic impedance.

In an embodiment, the S3, before the evaluating deviations of the strengths of the respective parts from the structural compressive strength, may further include: establishing an evaluation three-dimensional data model, storing three-dimensional data of the evaluation three-dimensional data model as array data by using spatial positions as unique identifiers (IDs). The evaluating deviations of the strengths of the respective parts from the structural compressive strength, specifically includes: evaluating the deviations of the strengths of the respective parts from the structural compressive strength being a global compressive strength, by using the three-dimensional data model.

In an embodiment, the S3 may further include: outputting an actual deviation numerical value of one of the parts of the fiber and nanosized materials reinforced concrete test block deviating beyond a deviation threshold, and corrective measures, according to a result of the evaluating deviations of the strengths of the respective parts from the structural compressive strength.

In an embodiment, the method may further include: S4, obtaining a color distribution image of impacted positions of the fiber and nanosized materials reinforced concrete test block by using a pressure-sensitive paper, and determining a damage degree of the fiber and nanosized materials reinforced concrete test block based on shadow distribution and shadow darkness in the color distribution image.

An equipment for evaluating a structural strength of a fiber and nanosized materials reinforced concrete, including:

an acquisition device (also referred to as acquisition module) configured for acquiring a sound velocity of a fiber and nanosized materials reinforced concrete test block;

a derivation device (also referred to as derivation module) configured for deriving a structural compressive strength of the concrete test block according to the sound velocity of the fiber and nanosized materials reinforced concrete test block and a relation between a fiber and nanosized materials reinforced concrete structural compressive strength and a fiber and nanosized materials reinforced concrete sound velocity;

a determination device (also referred to as judging module) configured for determining strengths of various parts of the fiber and nanosized materials reinforced concrete test block based on measured parameters and recorded design parameters of the fiber and nanosized materials reinforced concrete test block, and evaluating deviations of the strengths of the respective parts from the structural compressive strength.

In an embodiment, the acquisition device may include:

a first receptor (also referred to as first receiving unit or first receiver) configured for receiving a first echo amplitude of a first sound wave signal after being reflected by an interface between a sound transmission medium and air back to reach a sound source, when the sound transmission medium is not in contact with the fiber and nanosized materials reinforced concrete test block;

a second receptor (also referred to as second receiving unit or second receiver) configured for receiving a second echo amplitude of a second sound wave signal after being reflected by an interface between the sound transmission medium and the fiber and nanosized materials reinforced concrete test block, when the sound transmission medium is in contact with the fiber and nanosized materials reinforced concrete test block;

a first calculator (also referred to as first calculation unit) configured for calculating an acoustic pressure reflection coefficient of the interface between the sound transmission medium and the fiber and nanosized materials reinforced concrete test block based on the first echo amplitude and the second echo amplitude;

a second calculator (also referred to as second calculation unit) configured for calculating a characteristic impedance of the fiber and nanosized materials reinforced concrete test block according to the acoustic pressure reflection coefficient, and calculating the sound velocity of the fiber and nanosized materials reinforced concrete test block according to the characteristic impedance.

A non-transitory computer readable medium stored with instructions executable by a processor to carry out the structural strength evaluation method.

According to the structural strength evaluation method by the disclosure, the strength of the fiber and nanosized materials reinforced concrete is identified and intelligently analyzed to judge the strength of the fiber and nanosized materials reinforced concrete structure; judging whether the strength of fiber and nanosized materials reinforced concrete meets the design requirements may evaluate the design strength of each part and the deviation from the structural tensile strength and give corrective measures, and may also evaluate the overall toughness of some projects. In addition, the disclosure may evaluate the quality of the test block under the test results after objective improvement and the objective improvement method, and has excellent application prospects.

### BRIEF DESCRIPTION OF DRAWINGS

FIG. 1 shows a flowchart of a structural strength evaluation method of a fiber and nanosized materials reinforced concrete.

FIG. 2 is a schematic structural diagram of a structural strength evaluation equipment of the fiber and nanosized materials reinforced concrete.

### DETAILED DESCRIPTION OF EMBODIMENTS

The disclosure will be further described in detail below through specific embodiments in combination with the accompanying drawings.

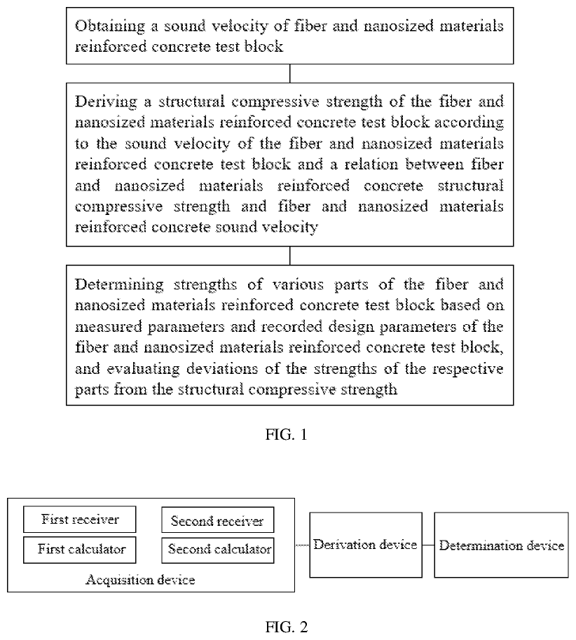

As shown in FIG. 1, the disclosure provides a structural strength evaluation method of fiber and nanosized materials reinforced concrete, specifically including:

a structural strength evaluation method of a fiber and nanosized materials reinforced concrete, including:

S1: obtaining a sound velocity of a fiber and nanosized materials reinforced concrete test block;

S2: deriving a structural compressive strength of the fiber and nanosized materials reinforced concrete test block according to the sound velocity of the fiber and nanosized materials reinforced concrete test block and a relation between a fiber and nanosized materials reinforced concrete structural compressive strength and a fiber and nanosized materials reinforced concrete sound velocity; and

S3: determining strengths of various parts of the fiber and nanosized materials reinforced concrete test block quickly based on measured parameters and recorded design parameters of the fiber and nanosized materials reinforced concrete test block by an on-site rebound method, and evaluating deviations of the strengths of the respective parts from the structural compressive strength.

In an embodiment, the sound velocity of the fiber and nanosized materials reinforced concrete test block is obtained based on an acoustic impedance method, specifically including the following steps:

coupling a sound source with a sound transmission medium under a condition that the sound transmission medium is not in contact with the fiber and nanosized materials reinforced concrete test block, controlling the sound source to transmit a first sound wave signal with predetermined frequency, amplitude and waveform to the sound transmission medium, and receiving a first echo amplitude of the first sound wave signal after being reflected by an interface between the sound transmission medium and air back to reach the sound source;

coupling the sound source with the sound transmission medium under a condition that the sound transmission medium is in contact with the fiber and nanosized materials reinforced concrete test block, controlling the sound source to transmit a second sound wave signal with the predetermined frequency, amplitude and waveform to the sound transmission medium, and receiving a second echo amplitude of the second sound wave signal after being reflected by an interface between the sound transmission medium and the fiber and nanosized materials reinforced concrete test block back to reach the sound source;

calculating an acoustic pressure reflection coefficient of the interface between the sound transmission medium and the fiber and nanosized materials reinforced concrete test block according to the first echo amplitude and the second echo amplitude;

calculating a characteristic impedance of the fiber and nanosized materials reinforced concrete test block according to the acoustic pressure reflection coefficient, and calculating the sound velocity of the fiber and nanosized materials reinforced concrete test block according to the characteristic impedance.

In an embodiment, the S3, before the evaluating deviations of the strengths of the respective parts from the structural compressive strength, may further include: establishing an evaluation three-dimensional data model, storing three-dimensional data of the evaluation three-dimensional data model as array data by using spatial positions as unique identifiers (IDs), and the evaluating deviations of the strengths of the respective parts from the structural compressive strength, specifically includes: using the evaluation three-dimensional data model to evaluate the deviations of the strengths of the respective parts from the structural compressive strength being as a global compressive strength. Moreover, the S3 may further include: outputting an actual deviation numerical value of one of the parts of the fiber and nanosized materials reinforced concrete test block deviating beyond a deviation threshold, and corrective measures, according to a result of the evaluating deviations of the strengths of the respective parts from the structural compressive strength.

Accordingly, the input mode of measured parameters is as follows: inputting the measured parameters of each partial compressive strength measurement under the evaluation three-dimensional data model, and storing the measured parameters as array data corresponding to the three-dimensional data. The input method of recorded design parameters is as follows: inputting the recorded design parameters of each partial compressive strength under the evaluation three-dimensional data model and storing the recorded design parameters as array data corresponding to the three-dimensional data.

In an embodiment, the method in this disclosure also includes using a pressure-sensitive paper to obtain a color distribution image of impact positions of the fiber and nanosized materials reinforced concrete test block, and determining a damage degree of the fiber and nanosized materials reinforced concrete test block based on shadow distribution and shadow darkness in the color distribution image.

The pressure-sensitive paper consists of two negative films coated with microcapsule chromogenic substances and chromogenic substances respectively. During the test, the coated parts are placed face to face. When the fiber and nanosized material reinforced concrete test block is impacted during the measurement process, the pressure-sensitive paper is stressed to cause the internal microcapsule to break and release chromogenic substances, and the chromogenic substances and chromogenic substances have a chromogenic reaction, thus generating colors; the color varies with the stress, so as to record the stress at different positions on the surface of the fiber and nanosized materials reinforced concrete test block with pressure-sensitive paper images.

Therefore, controlling the collection of the pressure at the impact position of the fiber and nanosized materials reinforced concrete test block is specifically realized through two-sided pressure-sensitive paper. During the test, the coating parts of the pressure-sensitive paper are placed face to face, the pressure-sensitive paper images are used to record the results of different positions on the surface of the concrete test block, and the obtained image information of the pressure-sensitive paper is converted into image digital information by the scanner, which becomes a two-dimensional pixel matrix recognizable by the program, and the image of damage degree is also scanned by the scanner. The internal program reads the shadow distribution and shadow darkness of the image digital information, identifies the pressure of the pressure-sensitive paper according to the established shadow distribution and shadow darkness-pressure correspondence, and then calculates and obtains the surface pressure distribution and the corresponding damage degree of the fiber and nanosized materials reinforced concrete test block at the impact position.

As shown in FIG. 2, the embodiment of the disclosure also provides an equipment for evaluating the structural strength of fiber and nanosized materials reinforced concrete, including:

an acquisition device (also referred to as acquisition module) configured for acquiring a sound velocity of a fiber and nanosized materials reinforced concrete test block;

a derivation device (also referred to as derivation module) configured for deriving a structural compressive strength of the fiber and nanosized materials reinforced concrete test block according to the sound velocity of the fiber and nanosized materials reinforced concrete test block and a relation between a fiber and nanosized materials reinforced concrete structural compressive strength and a fiber and nanosized materials reinforced concrete sound velocity;

a determination device (also referred to as determination module) configured for determining strengths of various parts of the fiber and nanosized materials reinforced concrete test block based on measured parameters and recorded design parameters of the fiber and nanosized materials reinforced concrete test block, and evaluating deviations of the strengths of the respective parts from the structural compressive strength. As an exemplary embodiment, the derivation device and the determination device are software modules stored in one or more memories and executable by one or more processors coupled to the one or more memories, in other words, are software modules stored in and executable by a computer system.

In an embodiment, the acquisition device includes:

a first receptor (also referred to as first receiving unit or first receiver) configured for receiving a first echo amplitude of a first sound wave signal after being reflected by an interface between a sound transmission medium and air back to reach a sound source, when the sound transmission medium is not in contact with the fiber and nanosized materials reinforced concrete test block;

a second receptor (also referred to as second receiving unit or second receiver) configured for receiving a second echo amplitude of a second sound wave signal after being reflected by an interface between the sound transmission medium and the fiber and nanosized materials reinforced concrete test block, when the sound transmission medium is in contact with the fiber and nanosized materials reinforced concrete test block;

a first calculator (also referred to as first calculation unit) configured for calculating an acoustic pressure reflection coefficient of the interface between the sound transmission medium and the fiber and nanosized materials reinforced concrete test block based on the first echo amplitude and the second echo amplitude; and

a second calculator (also referred to as second calculation unit) configured for calculating a characteristic impedance of the fiber and nanosized materials reinforced concrete test block according to the acoustic pressure reflection coefficient, and calculating the sound velocity of the fiber and nanosized materials reinforced concrete test block according to the characteristic impedance. As an exemplary embodiment, the first calculator and the second calculator are software modules stored in one or more memories and executable by one or more processors coupled to the one or more memories, in other words, are software modules stored in and executable by a computer system.

Some embodiments of the disclosure also provide a non-transitory computer readable medium on which instructions are stored, and the instructions, when executed by a processor, the structural strength evaluation method of fiber and nanosized materials reinforced concrete may be carry out.

The above embodiments may be implemented in whole or in part by software, hardware, firmware, or any combination thereof. When implemented using software, the implementation may be in whole or in part in the form of a computer program product. The computer program product comprises one or more computer instructions. When the computer loads and executes the computer program instructions, all or part of them produce a process or function as described in accordance with embodiments of the present application. The computer may be a general purpose computer, a specialized computer, a computer network, access to other programmable devices. The computer instructions may be stored in a computer readable medium or transmitted from one computer readable medium to another computer readable medium. For example, the computer instructions may be transmitted from one site, computer, server, or data center to another site via wired (e.g., coaxial cable, fiber optic, digital subscriber line (DSL)) or wireless (e.g., infrared, wireless, microwave, etc.) means the computer-readable medium can be any available medium accessible by a computer or a data storage device such as a server, data center, etc. that contains one or more available media integrated. The available media may be magnetic media, (e.g., floppy disk, hard disk, magnetic tape), optical media (e.g., DVD), or semiconductor media (e.g., Solid State Disk (SSD)), etc.

These skilled in the art should further realize that the units and algorithmic steps of each example described in conjunction with the embodiments disclosed in this disclosure may be implemented in electronic hardware, computer software, or a combination of both, and that the components and steps of each example have been described in the above description in general terms by function in order to clearly illustrate the interchangeability of hardware and software. Whether these functions are performed in hardware or software depends on the particular application and preset constraints of the technical solution. The skilled person may use different methods to implement the described functions for each particular application, but such implementation should not be considered outside the scope of this application.

It can be understood by those skilled in the art that all or part of the steps in the method of implementing the above embodiments can be instructed by the processor through a program, which can be stored in a computer-readable medium, which is a non-transitory medium, such as random access memory, read-only memory, flash memory, hard disk, solid state disk, magnetic tape floppy disk, optical disk and any combination thereof.

The above is only the preferred embodiment of this application, but the scope of protection of this application is not limited to this. Any changes or substitutions that can be easily thought of by those skilled in this field within the technical scope disclosed in this application should be covered by this application. Therefore, the scope of protection of this application should be subject to the scope of protection of the claims.

What is claimed is:

1. A structural strength evaluation method of fiber and nanosized materials reinforced concrete, comprising:
S1: obtaining a sound velocity of fiber and nanosized materials reinforced concrete test block;
S2: deriving a structural compressive strength of the fiber and nanosized materials reinforced concrete test block according to the sound velocity of the fiber and nanosized materials reinforced concrete test block and a relation between fiber and nanosized materials reinforced concrete structural compressive strength and fiber and nanosized materials reinforced concrete sound velocity; and
S3: determining strengths of various parts of the fiber and nanosized materials reinforced concrete test block based on measured parameters and recorded design parameters of the fiber and nanosized materials reinforced concrete test block, and evaluating deviations of the strengths of the respective parts from the structural compressive strength.

2. The structural strength evaluation method according to claim 1, wherein the sound velocity of the fiber and nanosized materials reinforced concrete test block is obtained based on an acoustic impedance method, specifically comprising:
coupling a sound source with a sound transmission medium under a condition that the sound transmission medium is not in contact with the fiber and nanosized materials reinforced concrete test block, controlling the sound source to transmit a first sound wave signal with predetermined frequency, amplitude and waveform to the sound transmission medium, and receiving a first echo amplitude of the first sound wave signal after being reflected by an interface between the sound transmission medium and air back to reach the sound source;
coupling the sound source with the sound transmission medium under a condition that the sound transmission medium is in contact with the fiber and nanosized materials reinforced concrete test block, controlling the sound source to transmit a second sound wave signal with the predetermined frequency, amplitude and waveform to the sound transmission medium, and receiving a second echo amplitude of the second sound wave signal after being reflected by an interface between the sound transmission medium and the fiber and nanosized materials reinforced concrete test block back to reach the sound source;
calculating an acoustic pressure reflection coefficient of the interface between the sound transmission medium and the fiber and nanosized materials reinforced concrete test block according to the first echo amplitude and the second echo amplitude; and
calculating a characteristic impedance of the fiber and nanosized materials reinforced concrete test block according to the acoustic pressure reflection coefficient, and calculating the sound velocity of the fiber and nanosized materials reinforced concrete test block according to the characteristic impedance.

3. The structural strength evaluation method according to claim 1, wherein the S3, before the evaluating deviations of the strengths of the respective parts from the structural compressive strength, further comprises:
establishing an evaluation three-dimensional data model, storing three-dimensional data of the evaluation three-dimensional data model as array data by using spatial positions as unique identifiers (IDs);
wherein the evaluating deviations of the strengths of the respective parts from the structural compressive strength, specifically comprises: evaluating the deviations of the strengths of the respective parts from the structural compressive strength being as a global compressive strength, by using the evaluation three-dimensional data model.

4. The structural strength evaluation method according to claim 1, wherein the S3 further comprises:
outputting an actual deviation value of one of the parts of the fiber and nanosized materials reinforced concrete test block beyond a deviation threshold, and corrective measures, according to a result of the evaluating deviations of the strengths of the respective parts from the structural compressive strength.

5. The structural strength evaluation method according to claim 1, further comprising:
S4, obtaining a color distribution image of impacted positions of the fiber and nanosized materials reinforced concrete test block by using a pressure-sensitive paper, and determining a damage degree of the fiber and nanosized materials reinforced concrete test block based on shadow distribution and shadow darkness in the color distribution image.

6. An equipment for evaluating a structural strength of fiber and nanosized materials reinforced concrete, comprising:
an acquisition device, configured for acquiring a sound velocity of a fiber and nanosized materials reinforced concrete test block;
a derivation device, configured for deriving a structural compressive strength of the fiber and nanosized materials reinforced concrete test block according to the sound velocity of the fiber and nanosized materials reinforced concrete test block and a relation between fiber and nanosized materials reinforced concrete structural compressive strength and fiber and nanosized materials reinforced concrete sound velocity; and
a determination device, configured for determining strengths of various parts of the fiber and nanosized materials reinforced concrete test block based on measured parameters and recorded design parameters of the fiber and nanosized materials reinforced concrete test block, and evaluating deviations of the strengths of the respective parts from the structural compressive strength.

7. The equipment according to claim 6, wherein the acquisition device comprises:
a first receiver, configured for receiving a first echo amplitude of a first sound wave signal after being reflected by an interface between a sound transmission medium and air back to reach a sound source, when the sound transmission medium is not in contact with the fiber and nanosized materials reinforced concrete test block;
a second receiver, configured for receiving a second echo amplitude of a second sound wave signal after being reflected by an interface between the sound transmission medium and the fiber and nanosized materials reinforced concrete test block, when the sound transmission medium is in contact with the fiber and nanosized materials reinforced concrete test block;
a first calculator, configured for calculating an acoustic pressure reflection coefficient of the interface between the sound transmission medium and the fiber and nanosized materials reinforced concrete test block based on the first echo amplitude and the second echo amplitude; and
a second calculator, configured for calculating a characteristic impedance of the fiber and nanosized materials reinforced concrete test block according to the acoustic pressure reflection coefficient, and calculating the sound velocity of the fiber and nanosized materials reinforced concrete test block according to the characteristic impedance.

8. A non-transitory computer readable medium stored with instructions executable by a processor to carry out the structural strength evaluation method of claim 1.
